(19) United States
(12) Patent Application Publication
(10) Pub. No.: US 2025/0217985 A1
(43) Pub. Date: Jul. 3, 2025

(54) METHOD AND SYSTEM FOR DETERMINING A TREATMENT STRATEGY FOR A VESSEL WITH MULTIPLE OR DIFFUSE LESIONS

(71) Applicant: MEDHUB LTD, Tel Aviv (IL)

(72) Inventors: OR BRUCH EL, RAMAT GAN (IL); YARDEN BRUCH EL, RAMAT GAN (IL); ALEXANDRE KASSEL, NATANYA (IL); SEFI FUCHS, GIVATAYIM (IL)

(21) Appl. No.: 19/006,337

(22) Filed: Dec. 31, 2024

(30) Foreign Application Priority Data
Dec. 31, 2023 (IL) 309861

(51) Int. Cl. G06T 7/00; A61B 34/10; G06V 10/62; G16H 30/40; G16H 50/50

(52) U.S. Cl. CPC G06T 7/0014; G06V 10/62; G16H 30/40; G16H 50/50; A61B 34/10; A61B 2034/105; G06T 2207/10016; G06T 2207/20081; G06T 2207/30096; G06T 2207/30101; G06V 2201/03

(57) ABSTRACT

Non-invasively determining a treatment strategy for a blood vessel by obtaining a prediction of a total pressure drop value in the blood vessel based on features generated from a plurality of images of the blood vessel, the images representing all anatomic parts of the blood vessel, and calculating a contribution of one or more portion(s) of the blood vessel to the total pressure drop value. Based on the calculated contribution, a new simulated total pressure drop value of the blood vessel is calculated by neutralizing the contribution of the one or more portion(s) to the total pressure drop value to determine if the new simulated pressure drop improves, thereby indicating portions which should be treated to restore healthy pressure drop values.

### FIELD

The invention relates generally to automated analysis of vessels, and particularly to blood vessels having multiple or diffuse lesions.

### BACKGROUND

Coronary artery disease (CAD) is typically caused by lesions or stenoses, which are local narrowings in coronary arteries, limiting blood flow to the heart muscle. Especially challenging CAD may include tandem lesions, which are multiple stenoses in series along a coronary artery and/or diffuse lesions characterized by multiple or especially long, continuous plaques or other lesions within coronary arteries.

Severeness of a stenosis or lesion can be assessed by measuring pressure differences across the stenosis in a technique called fractional flow reserve (FFR). FFR is defined as the pressure after (distal to) a stenosis relative to the pressure before the stenosis, under hyperemia, thus expressing the maximal flow down a vessel in the presence of a stenosis compared to the maximal flow in the hypothetical absence of the stenosis. Sever stenoses or lesions are treated by angioplasty and placement of stents, however, diagnosing and treating diffuse and tandem lesions is challenging.

Pressure measurements enable detailed assessment of the physiological significance of a stenosis but in the presence of multiple or diffused lesions, predicting the impact of stenting a given stenosis can be difficult and is impeded by flow interaction between stenoses under hyperemia. In the situation of a vessel with two tandem lesions, hyperemic flow across the proximal lesion is a function not only of the stenosis geometry but also of the effect of the second, distal lesion. Similarly, hyperemic flow across the second lesion is a function of its specific geometry and its inlet flow, which is influenced by the proximal stenosis. As a result, it is not possible to determine an accurate FFR for each individual stenosis in a case of diffuse disease.

A technique called instantaneous wave-free ratio or instant flow reserve (iFR) isolates a specific period in diastole (when the heart is at rest), called the wave-free period, during which the coronary artery is least affected by the pulsatile blood flow and the competing forces (waves) that affect coronary flow are quiescent, meaning pressure and flow are linearly related as compared to the rest of the cardiac cycle. After obtaining a baseline iFR value at a location of interest, iFR measurements at multiple points along the vessel are obtained and can be plotted throughout the vessel to create a pullback curve. The pullback curve can assist in identifying pressure gradients along the artery, can help identify focal and diffuse coronary disease and can be used to assess the physiological significance of a stenosis even in cases of tandem lesions or diffuse disease.

iFR pullback is typically performed using invasive coronary pressure wires which are placed in the coronary artery and are slowly withdrawn (pulled back), when the heart is at rest. This invasive procedure is typically time-consuming and requires high expertise.

Recently emerging non-invasive methods for assessment of coronary artery disease are typically based on flow dynamics analysis from image data of a patient's coronary arteries. One other suggested non-invasive method includes using trained models to compute a hemodynamic quantity of interest at a plurality of points along the coronary artery tree. Computing the hemodynamic quantity of interest at points along healthy segments of the coronary artery tree is done using a first trained regression model and computing the hemodynamic quantity of interest at the multiple points within each of one or more lesions, is done using a second trained regression model. A pullback curve is then generated based on the computed hemodynamic quantity of interest at the multiple points along the artery.

### SUMMARY

Embodiments of the invention provide methods and systems for non-invasively determining a treatment strategy for a blood vessel with multiple or diffuse lesions, without calculating pressure measurements at a plurality of points along a patient's vessel and without relying on a pullback curve whose analysis requires time and expertise.

A method according to one embodiment of the invention includes obtaining a prediction of a total pressure drop value of the blood vessel based on features generated from a plurality of images of the blood vessel. The features may be related to image data and/or may be related to specific portions of the images, e.g., to portions of the blood vessel featured in the image. The contribution of one or more portion(s) of the blood vessel to the total pressure drop value is calculated and based on the calculated contribution, a new simulated total pressure drop value of the blood vessel is calculated, by neutralizing the contribution of the one or more portion to the total pressure drop. The new simulated total pressure drop value and, typically, an indication of the one or more portion whose contribution to the total pressure drop has been neutralized, may be displayed to a user.

Typically, if a portion of a vessel has a high contribution to a total pressure drop in the vessel, this may indicate that the portion includes a sever lesion or lesions. Neutralizing the contribution of this portion will typically provide a simulated pressure drop which is reduced compared with the initially predicted total pressure drop, possibly indicating that this portion should be treated (e.g., stented). This automatic calculation of the contribution of a portion of a blood vessel to the total pressure drop, provides, quickly and simply, an indication of which portions of a vessel should be treated in order to reduce the total pressure drop predicted for the vessel. Thus, a user may be able to determine an appropriate treatment strategy without having to do any time-consuming and expert analyses of a plurality of pressure measurements and of a pull-back curve.

### BRIEF DESCRIPTION OF THE DRAWINGS

The invention will now be described in relation to certain examples and embodiments with reference to the following illustrative drawing figures so that it may be more fully understood. In the drawings:

FIG. 1 schematically illustrates a method for non-invasively determining a treatment strategy for a blood vessel with multiple or diffuse lesions, according to embodiments of the invention;

FIG. 2 schematically illustrates a method for non-invasively determining a treatment strategy for a blood vessel with multiple or diffuse lesions, according to other embodiments of the invention;

FIG. 3 schematically illustrates a method for non-invasively determining a treatment strategy for a blood vessel with multiple or diffuse lesions, according to yet other embodiments of the invention;

FIG. 4 schematically illustrates a method for non-invasively determining a treatment strategy for a blood vessel with multiple or diffuse lesions, based on a prior determination of the presence of multiple or diffuse lesions, according to embodiments of the invention;

FIG. 5 schematically illustrates a method for non-invasively determining a treatment strategy for a blood vessel with multiple or diffuse lesions, using image data combined from a plurality of images, each image captured from a different angle, according to embodiments of the invention;

FIG. 6 schematically illustrates a method for non-invasively determining a treatment strategy for a blood vessel with multiple or diffuse lesions, using optimal images, according to embodiments of the invention;

FIG. 7A schematically illustrates a system for non-invasively determining a treatment strategy for a blood vessel with multiple or diffuse lesions, according to embodiments of the invention;

FIG. 7B schematically illustrates a machine learning module, according to embodiments of the invention, that may be part of the system illustrated in FIG. 7A;

FIG. 8 schematically illustrates a fully automated process, according to an embodiment of the invention; and

FIG. 9 schematically illustrates a user interface that may be part of a system for non-invasively determining a treatment strategy, according to embodiments of the invention.

### DETAILED DESCRIPTION

Embodiments of the invention provide methods and systems for automated analysis of vessels from images of the vessels to obtain a prediction of a functional measurement of the vessel and/or portions of the vessel. In one embodiment, a total pressure drop value in the blood vessel (namely, the pressure drop across the length of the vessel or from a proximal point to a distal point) is predicted. A prediction of a contribution (e.g., a numerical contribution) of a part of the analyzed vessel to the total pressure drop value is generated and information regarding the contribution is displayed to a user (e.g., a physician or technician), to enable determining a treatment strategy for the vessel. As described above, if a portion of a vessel has a high contribution to a total pressure drop in the vessel and neutralizing the contribution of this portion provides a reduced total pressure drop, possibly a physiologically healthy pressure drop, this may indicate that treatment of this portion can restore the pressure drop in the vessel to a physiologically healthy level. Thus, a user may be able to determine an appropriate treatment strategy by obtaining information automatically provided by methods and systems of the invention and without having to do any time-consuming and expert analyses of a plurality of pressure measurements and of a pull-back curve.

Analysis, according to embodiments of the invention, may include calculations and predictions described herein as well as applying algorithms to obtain diagnostic information, such as presence of a pathology, identification of the pathology, location of the pathology, etc.

A “vessel” may include a tube or canal in which body fluid is contained and conveyed or circulated. Thus, the term vessel may include blood veins or arteries, coronary blood vessels, lymphatics, portions of the gastrointestinal tract, etc.

An image of a vessel may be obtained using suitable imaging techniques, for example, X-ray imaging, ultrasound imaging, Magnetic Resonance imaging (MRI) and others suitable imaging techniques. Some embodiments of the invention use angiography, which includes injecting a radio-opaque contrast agent into a patient's blood vessel and imaging the blood vessel using X-ray based techniques. The images used, according to embodiments of the invention, are typically 2D lengthwise images of a vessel, as opposed to, for example, 2D cross section images that are used in methods that require constructing a 3D model of the vessel, such as coronary tomography angiography (CTA) and other computerized tomography methods.

A pathology may include, for example, a narrowing of the vessel (e.g., stenosis or stricture), lesions within the vessel, etc.

A “functional measurement” is a measurement of the effect of a pathology on flow through the vessel. Functional measurements may include measurements such as an estimate of fractional flow reserve (FFR), an estimate of instant flow reserve (iFR), coronary flow reserve (CFR), quantitative flow ratio (QFR), resting full-cycle ratio (RFR), quantitative coronary analysis (QCA), and more.

No local pressure values are measured or estimated in embodiments of the invention, however, a pressure drop between two points in a vessel is estimated (e.g., predicted). Thus, methods and systems according to embodiments of the invention may mimic iFR measurements without invasive procedures and without generating a pullback curve, which is often a cause of grievance to physicians.

In the following description, various aspects of the invention will be described. For purposes of explanation, specific configurations and details are set forth in order to provide a thorough understanding of the invention. However, it will also be apparent to one skilled in the art that the invention may be practiced without the specific details presented herein. Furthermore, well known features may be omitted or simplified in order not to obscure the invention.

Unless specifically stated otherwise, as apparent from the following discussions, it is appreciated that throughout the specification discussions utilizing terms such as “using”, “analyzing”, “processing,” “computing,” “calculating,” “estimating”, “predicting”, “generating”, “determining,” “detecting”, “identifying” or the like, refer to the action and/or processes of a computer or computing system, or similar electronic computing device, that manipulates and/or transforms data represented as physical, such as electronic, quantities within the computing system's registers and/or memories into other data similarly represented as physical quantities within the computing system's memories, registers or other such information storage, transmission or display devices. Unless otherwise stated, these terms refer to automatic action of a processor, independent of and without any actions of a human operator.

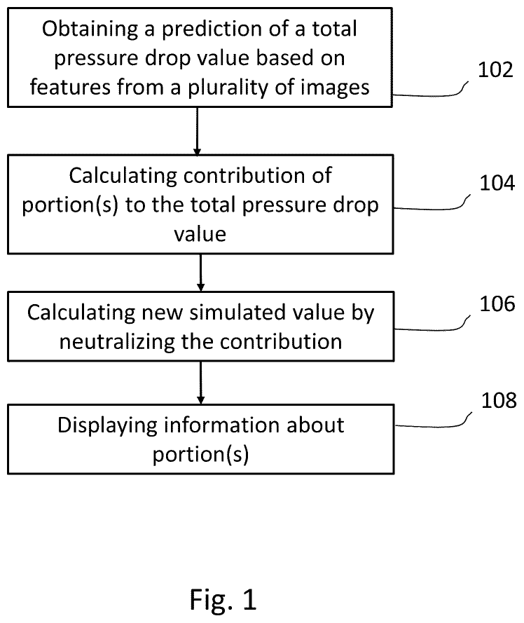

In one embodiment, which is schematically illustrated in FIG. 1, a method for non-invasively determining a treatment strategy for a blood vessel, possibly, with multiple or diffuse lesions, is exemplified. The method includes no steps of determining pressure at any (one or multiple) locations along the vessel and does not rely on a pullback curve or graph being created or displayed to a user. In one embodiment, the method includes obtaining a prediction of a total pressure drop value in a blood vessel based on features generated from a plurality of images of the blood vessel (step 102). The features may be, for example, spatial and/or temporal features.

The entire blood vessel that is being analyzed is represented in the images. Namely, the images capture all of the anatomic parts of the blood vessel for which a pressure drop is being predicted. Typically, the plurality of images are 2D lengthwise images of the vessel, typically captured using X-ray imaging in an angiogram procedure. Angiogram videos may be captured from different views or angles and the plurality of images may include individual frames each taken from a different angiogram video, such that each of the plurality of images is captured from a different angle. The term “video” is used herein to mean any type of sequence of frames or images.

The images may be obtained and analyzed on-line, e.g., the images may be obtained directly from an imaging device being used during a procedure (e.g., angiography). The images may also be analyzed off-line, e.g., in a physician's office. For example, the images may be part of a DICOM (digital imaging and communications in medicine) file or any other format used for storage and exchange of medical images, as well as related information, e.g., metadata such as patient information and imaging parameters. DICOM files may be used off-line to provide the images to be analyzed according to embodiments of the invention.

In step 104, a contribution of one or more portion(s) of the blood vessel to the total pressure drop value, is calculated and in step 106, using the calculated contribution, a new simulated total pressure drop value of the blood vessel is calculated, by neutralizing the contribution of the one or more portion to the total pressure drop value. For example, a total pressure drop value may be calculated by disregarding the component representing the one or more portion whose contribution is being neutralized or by assigning to it a value of zero or null.

The one or more portions may be predetermined portions. For example, each portion may be a different anatomical part of the vessel (e.g., proximal, medial and distal). Alternatively or in addition, the portions may be defined by detected pathologies. For example, a portion may be defined from one pathology to another, or a portion may be defined as a part that includes both healthy and diseased parts (e.g., parts containing at least one pathology) of the vessel.

In step 108 information about the one or more portion(s) is displayed to the user. The displayed information may include the new simulated total pressure drop value and/or an indication of the one or more portion whose contribution was neutralized. In some embodiments, the predicted total pressure drop value may also be displayed to the user. In some embodiments, the information may be displayed to the user via a graphical presentation (e.g., including a diagram, graph and/or chart) that shows the contribution to the total pressure drop of the different portions and/or pathologies with respect to their locations along the vessel. Other graphical presentations may be displayed to the user.

In some embodiments, the method may include displaying to the user a cluster of images featuring a same pathology (e.g., the same lesion or other diseased area), each of the images in the cluster being captured from a different angle. The indication of the one or more portion whose contribution was neutralized may be superimposed on the displayed cluster (e.g., on one or more images making up a cluster), for example, a graphic mark may be superimposed on one or more images of the displayed cluster.

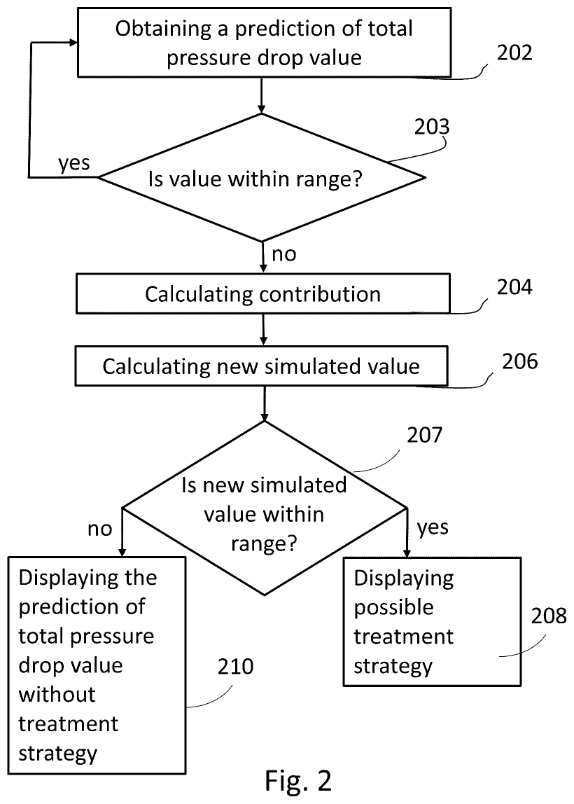

Another embodiment of the invention is schematically illustrated in FIG. 2. In the embodiment exemplified in FIG. 2, a method for non-invasively determining a treatment strategy for a blood vessel, possibly with multiple or diffuse lesions, may include determining if the new simulated total pressure drop value is in a physiologically healthy range (e.g., above or below a threshold) and displaying to the user the new simulated total pressure drop value and/or a suggestion for a treatment strategy (e.g., displaying an indication of the one or more portion whose contribution to the total pressure drop was neutralized), only if the new simulated total pressure drop value is in the physiologically healthy range.

In step 202 a prediction of a total pressure drop value in a blood vessel is obtained, based on features generated from a plurality of images of the blood vessel, for example, as described with reference to FIG. 1 and further below.

In step 203 it is determined if the predicted total pressure drop value of the blood vessel is within a physiologically healthy range. For example, a physiologically healthy range may be determined by health organizations or determined by a physician or technician. In one example, a total pressure drop (e.g., the difference in the pressure value between a beginning point and an end point of a blood vessel or portion of the vessel) of less than 10% (namely, less than 0.1) may be regarded as a normal drop but a pressure drop larger than 10% may be indicative of a pathology. Thus, a physiologically healthy range may be between 0 to 0.1, whereas any total pressure drop value above 0.1 may be indicative of a pathology.

If the predicted total pressure drop value is within the physiologically healthy range, then there is no need for treatment. The predicted total pressure drop value (which is within the physiologically healthy range) may be displayed to a user and the method may go on to check another vessel or different portion of the vessel. If the total pressure drop value is not within the physiologically healthy range, then a contribution of one or more portion(s) of the blood vessel to the total pressure drop value, is calculated (in step 204) and using the calculated contribution, a new simulated total pressure drop value of the blood vessel is calculated (in step 206) by neutralizing the contribution of the one or more portion to the total pressure drop value. It is then determined which one or more portion's contribution, when neutralized, produces a new simulated total pressure drop value that is within the physiologically healthy range (e.g., below 0.1). If, in step 207, the one or more portion's contribution, when neutralized, produces a new simulated total pressure drop value that is within the physiologically healthy range, then a possible treatment strategy may be displayed to a user (in step 208), which typically includes an indication of the one or more portions whose contribution was neutralized, to provide a user with an indication of which portion should be treated to reduce the total pressure drop value to a physiologically healthy level.

If, in step 207, the one or more portion's contribution, when neutralized, produces a new simulated total pressure drop value that is not within the physiologically healthy range (e.g., the new simulated value is above 0.1, which may correspond to an iFR value of below 0.9) then the method may go on to check the contribution of other portion(s) of the vessel.

If all portions of the vessel are analyzed (e.g., the contribution of each portion to the new simulated total pressure drop value (that is not within the physiologically healthy range) is neutralized) and a physiologically healthy total pressure drop value cannot be obtained, then the initially predicted total pressure drop value (that is not within the physiologically healthy range) is displayed without an indication of any portion, namely, the predicted total pressure drop value is displayed without a possible treatment strategy (step 210). In some embodiments, the predicted total pressure drop value being displayed in step 210 can be marked (e.g., by a color or other indication) as being not within the physiologically healthy range.

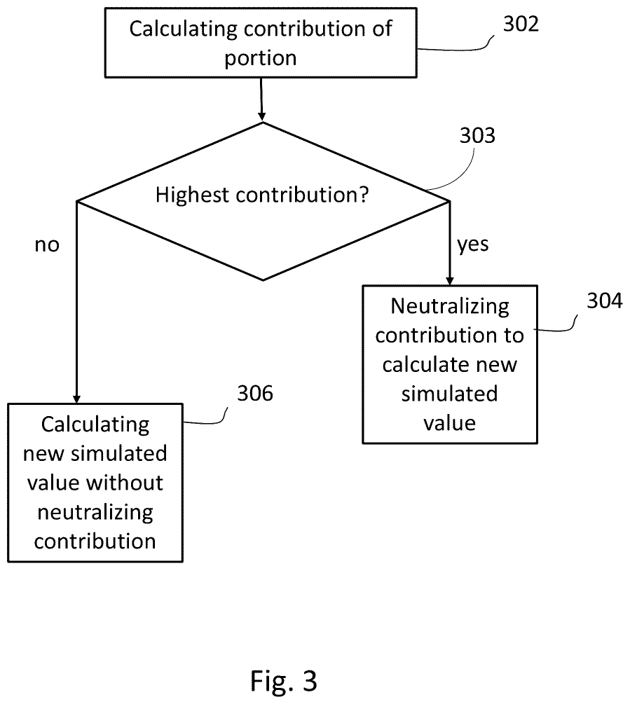

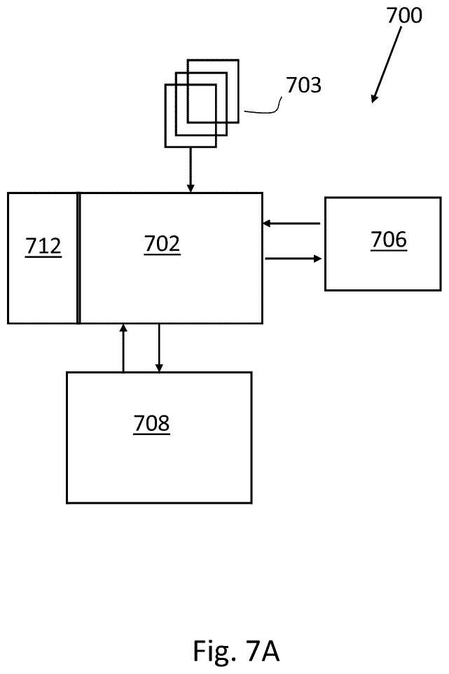

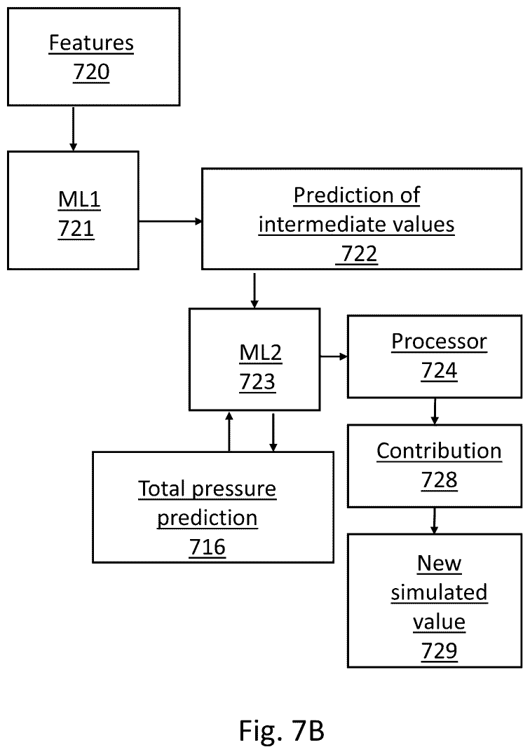

Another embodiment of the invention, which is schematically illustrated in FIG. 3, includes determining which portion of the vessel has the most significant contribution to the total pressure drop and then neutralizing the contribution of this portion to the total pressure drop. In the embodiment exemplified in FIG. 3, the contribution to the total pressure drop value of each of a plurality of portions of a patient's vessel, is calculated in step 302. In step 303 it is determined if a specific portion's contribution is most significant, namely, if the contribution of the portion to the total pressure drop value is highest. This determination may be done, for example, by using calculations of percentages or comparison of values. In some embodiments, this determination may be done by using a machine learning model, e.g., as further detailed below with reference to FIG. 7B.

If the specific portion is determined, in step 303, to have the most significant contribution, then a new simulated total pressure drop is calculated by neutralizing the contribution of this specific portion, in step 304. If the specific portion is determined not to have the most significant contribution, then the contribution of the specific portion is not neutralized, but rather used in the calculation of the new simulated total pressure drop, in step 306.

In some embodiments of the invention, the steps described above (e.g., with reference to FIGS. 1-3) are only or usually performed after the presence of diffuse disease or multiple lesions in the blood vessel is determined.

The presence of diffuse disease or multiple lesions in the blood vessel may be determined by tracking the blood vessel in images of the vessel, each image captured from a different view or angle. In one embodiment, a blood vessel can be marked by a user, e.g., by the user entering a mark of the vessel on a user interface on which an image of the blood vessel is displayed. Alternatively or in addition, computer vision algorithms may be used to identify a blood vessel in a first image. The blood vessel may then be marked, e.g., using a virtual mark, such that it can be tracked, namely, such that it can be easily detected in future images even if the future images are captured from a different angle than the angle of capture of the first image and have different visual characteristics than the first image. Similarly, a pathology (such as diffuse disease or multiple lesions) can be marked by a user or can be identified using computer vision techniques. The user-marked or identified pathology may then be marked, e.g., using a virtual mark (which can be, for example, location based, e.g., based on a location of the pathology in relation to portions or structures of the vessel), so that it can be tracked, namely, so that it can be easily identified in future images, even if the future images are captured from a different angle than the angle of capture of the first image and have different visual characteristics than the first image.

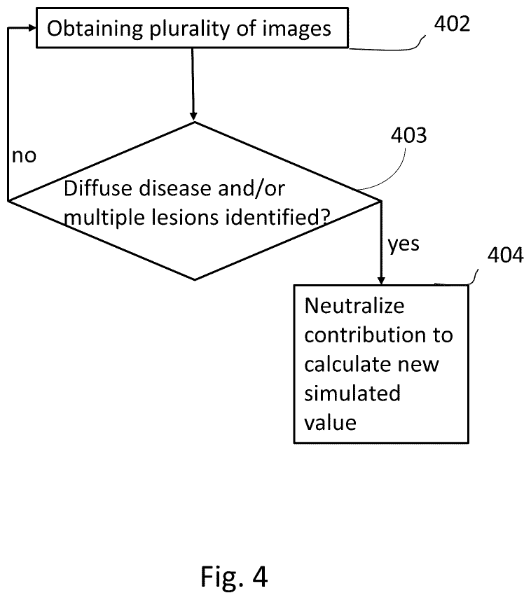

In the embodiment exemplified in FIG. 4, a plurality of images, each captured from a different angle, is obtained (in step 402). The plurality of images may include frames, each selected from one of a plurality of angiogram videos, each video captured from a different angle. A blood vessel may be identified and tracked through the plurality of images and pathologies may be identified in the blood vessel. If diffuse disease and/or multiple lesions are identified within the blood vessel (in step 403) then the method proceeds with the different calculations (in step 404) described above, e.g., the method proceeds to obtain a prediction of the contribution of each of a plurality of portions of the blood vessel to the total pressure drop and may proceed to provide the new simulated total pressure drop value and an indication of the one or more portion whose contribution was neutralized, as described above (e.g., with reference to FIG. 1). Thus, in this embodiment, the presence of diffuse disease or multiple lesions in the blood vessel is determined prior to commencing with any one of the different calculations described above, e.g., prior to obtaining a prediction of a total pressure drop value of the blood vessel or prior to obtaining a new simulated total pressure drop.

If diffuse disease and/or multiple lesions are not identified within the blood vessel (in step 403), then the method does not proceed with the calculations and a new set of images can be analyzed for the presence of pathologies.

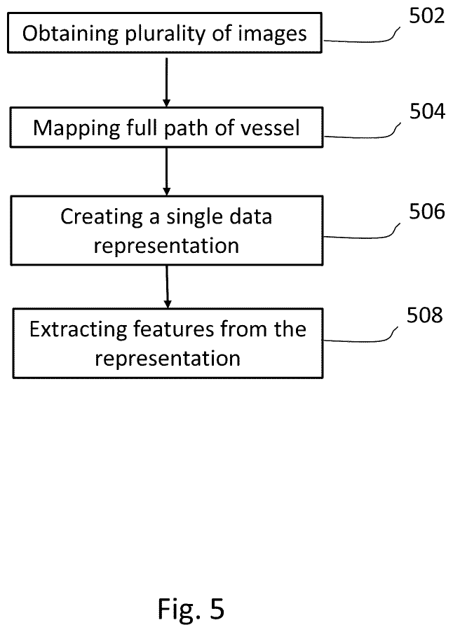

FIG. 5 schematically illustrates a method that uses data combined from a plurality of images, each image captured from a different angle, according to embodiments of the invention.

In step 502 a plurality of images, each captured from a different angle, is obtained. Each of the plurality of images may be selected, e.g., from one of a plurality of angiogram videos, each video captured from a different angle. A blood vessel may be identified and tracked through the plurality of images (for example by marking the identified blood vessel in each of the images, as described above). In step 504, a full path of the identified blood vessel is mapped. A “full path” typically refers to a length of the vessel which includes all of the pathologies that may contribute to the total pressure drop. For example, a full path of a left main coronary artery (LMCA) may include a path from a beginning point of the artery until after bifurcations of the artery or, for example, a full path of a right coronary artery (RCA) may include a path from a beginning point of the artery until the artery becomes too narrow to allow obtaining meaningful information from it. The mapping of a full path of each artery enables separately analyzing each artery.

Once a full path of the vessel is mapped, data from different-angle images (images captured from different angles) featuring the same vessel (e.g., as determined by tracking the vessel, as described above) is used, in step 506, to create a single data representation. In one embodiment, the single data representation may include data combined from the different-angle images. For example, different-angle images may be aggregated and transformed to a single coordinate image data representation, e.g., by registration of the images. In another embodiment, a single data representation may include non-image data. For example, a single data representation can be created based on alignment of 1-dimensional (1D) characteristics of the vessel (such as information about a specific location along the vessel, e.g., the width of the vessel at a location along the vessel or the presence of a pathology at a location along the vessel), to create a data representation which is a 1D signal. In other embodiments, the single data representation need not include the data (such as the combined data described above), rather, data from different-angle images may be used, e.g., mathematically manipulated (such as averaged), to create a single data representation that is based on data from different-angle images but need not include the data itself.

The single data representation can then be used in step 508 for extracting features from it. The extracted features, which may be for example, spatial and/or temporal features, may be used for obtaining a prediction of the total pressure drop value, e.g., as further described below, with reference to FIG. 7B.

Temporal features, for example, may be extracted from a signal created from an attribute of an image of the vessel, which is recorded over time. An attribute of an image of the vessel may be a visible characteristic of the image or other characteristic inherent to the image. For example, an attribute may include pixel intensity or pixel color or grey levels. In one example, an attribute may include the number of pixels having a color or intensity above a threshold and/or the distribution of these pixels.

In some embodiments, a temporal feature includes a calculation of a combination of attribute values determined from a plurality of images whereas spatial features may include, for example, visible attributes of images.

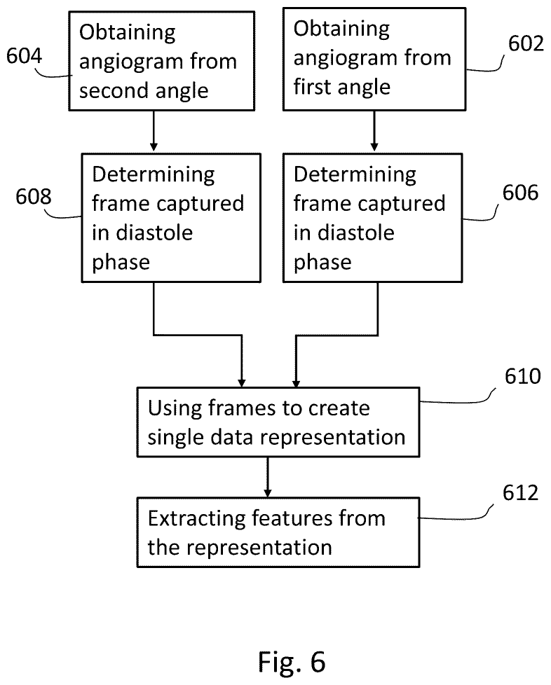

FIG. 6 schematically illustrates one possible method of selecting an image, e.g., from an angiogram video, as in steps 402 and/or 502 described above.

In step 602, a patient's first angiogram video captured from a first angle is obtained and in step 604 a second angiogram video (of the same patient and same location on the patient) captured from a second angle is obtained. Angiograms or images can be obtained from different angles, e.g., by moving the camera between two view points and/or by rotating the patient, e.g., on an angiography table.

In steps 606 and 608 it is determined which frames in the first angiogram video and second angiogram video (correspondingly) were captured at a diastole phase (rest) of a cardiac cycle of the patient. In step 610 frames that were determined to be captured at a diastole phase, are used to create a single data representation, e.g., as described above. In step 612 the single data representation can be used for extracting features from it (e.g., as described herein with reference to FIG. 5 and FIG. 7B).

Thus, frames that were captured at a diastole phase from a plurality of angiogram videos, each captured from a different angle, may be used as the plurality of images on which further calculations (e.g., obtaining predictions as described herein) are performed.

In some embodiments, determining which frames were captured at the diastole phase of the cardiac cycle includes selecting frames (from angiogram videos captured from different angles) with the highest density of contrast agent and then selecting frames featuring a vessel full of contrast agent. The selected frames are input into a ML model trained to find a significant point of interest in the cardiac cycle based on frames featuring a vessel full of contrast agent, each of the frames captured from a different angle, to select a frame captured at the diastole phase.

A significant point of interest in the cardiac cycle may include, for example, an R-peak as determined by an electrocardiogram (ECG).

A system for non-invasively determining a treatment strategy for a blood vessel with multiple or diffuse lesions, according to one embodiment of the invention, is schematically illustrated in FIG. 7A.

In one embodiment, system 700 includes a machine learning (ML) unit 708 to predict a total pressure drop value of a blood vessel from images of the blood vessel. Typically, the images include lengthwise 2D images captured from different angles (e.g., images selected from angiogram videos captured from different angles). The total pressure drop value is predicted without obtaining pressure measurements at locations along the vessel.

System 700, which can perform the methods or steps described herein, includes a processor 702 in communication with ML unit 708 and with a user interface device 706. Processor 702 receives one or more images 703 of a patient's vessel. The images 703 may include different-angle images, typically frames of angiogram videos, from which frames may be selected, e.g., as described herein.

A single data representation may be created from images 703 by processor 702 or by another processing unit. Features (e.g., spatial and/or temporal features) are extracted from the single data representation by processor 702 or by another processing unit. The extracted features may be input to ML unit 708. ML unit 708 may provide, based on the inputted features, a prediction of a total pressure drop value of a blood vessel featured in one or more of images 703 and/or a contribution of one or more portion(s) of the blood vessel to the total pressure drop and/or a new simulated total pressure drop value of the blood vessel which takes into account neutralization of the contribution of the one or more portion to the total pressure drop value. Some of the outputs of ML 708 can be calculated by processor 702 or other processing units.

A new simulated total pressure drop value and, optionally, an indication of the one or more portion whose contribution has been neutralized to obtain the new simulated total pressure drop value, can be displayed on user interface device 706, e.g., as part of a possible treatment strategy. In some embodiments, the total pressure drop value may also be displayed to a user, e.g., on user interface device 706.

Processor 702 may cause an indication of the one or more portion to be displayed, via the user interface device 706, on an image of the patient's vessels (e.g., image 703) that is also displayed. An indication of the one or more portions displayed on a display of user interface device 706 may include, for example, graphics, such as, letters, numerals, symbols, different colors and shapes, etc., that can be superimposed on the image or images of a video of the patient's vessels. Alternatively or in addition, an indication of the one or more portion may include a textual description on the portion, e.g., referring to an anatomic location or other anatomical features of the portion.

In some embodiments, user input can be received at processor 702, via user interface device 706. User interface device 706 may include a display, such as a monitor or screen, for displaying images, instructions and/or notifications to a user (e.g., via graphics, images, text or other content displayed on the monitor). User interface device 706 may also be designed to receive input from a user. For example, user interface device 706 may include or may be in communication with a mechanism for inputting data, such as a keyboard and/or mouse and/or touch screen, to enable a user to input data.

Processor 702 may include, for example, one or more processors and may be a central processing unit (CPU), a graphics processing unit (GPU), a digital signal processor (DSP), a field-programmable gate array (FPGA), a microprocessor, a controller, a chip, a microchip, an integrated circuit (IC), or any other suitable multi-purpose or specific processor or controller. Processor 702 and/or ML unit 708 may be locally embedded or remote, e.g., in the cloud.

Processor 702 is typically in communication with a memory unit 712. In one embodiment, memory unit 712 stores executable instructions that, when executed by the processor 702, facilitate performance of operations of the processor 702, as described herein. Memory unit 712 may also store image data (which may include data such as pixel values that represent the intensity of light having passed through body tissue and/or light reflected from tissue or from a contrast agent within vessels, and received at an imaging sensor, as well partial or full images or videos) of at least part of the images 703.

Memory unit 712 may include, for example, a random access memory (RAM), a dynamic RAM (DRAM), a flash memory, a volatile memory, a non-volatile memory, a cache memory, a buffer, a short term memory unit, a long term memory unit, or other suitable memory units or storage units.

All or some of the components of system 700 may be in wired or wireless communication, and may include suitable ports such as USB connectors and/or network hubs.

In one embodiment, image(s) 703 include a 2D lengthwise image of a patient's vessels (e.g., an angiogram image and/or video). Processor 702 receives one or more image(s) and uses ML techniques and/or additional computer vision algorithms to detect and identify vessels in the image 703. In one example, classifiers such as Random Forest Classifier or CNN classifiers may be pre-trained on training data that includes, e.g., 2D lengthwise X-ray angiogram images of anatomically classified vessels, to identify and anatomically classify arteries from the images. Anatomic classification of vessels may include classifying vessels based on their anatomic location (e.g., left/right) and/or other anatomical features, for example, right coronary artery (RCA), left anterior descending artery (LAD) and circumflex branch of the left coronary artery (LCX).

Processor 702 may further obtain an indication of a presence of a pathology (e.g., stenosis) in a vessel and possibly a location of the pathology, e.g., by applying a classifier on the image(s) 703. Classifiers, such as DenseNet, CNN (Convolutional Neural Network) or EfficientNet, may be used to obtain a determination of presence of a pathology and to determine the location of the pathology. Classifiers, according to embodiments of the invention, may be pre-trained on training data that includes 2D lengthwise X-ray angiogram images of vessels which may include a pathology (e.g., stenosis). The neural network composing the classifier may be learned by a scheme of supervised learning, or possibly semi-supervised learning.

Applying a classifier, using ML techniques and/or additional or other computer vision techniques on an angiography image enables automatic detection of a vessel and/or pathology and mapping the full length of a vessel, without requiring, e.g., user input.

In some embodiments, the lengthwise 2D image is an optimal image, selected from a plurality of 2D images of the patient's vessels, as the image showing the most detail. In the case of angiogram images, which include contrast agent injected to a patient to make vessels (e.g., blood vessels) visible on an X-ray image, an optimal image may be an image of a blood vessel showing a large/maximum amount of contrast agent. Thus, an optimal image can be detected by applying image analysis algorithms (e.g., to detect the image frames having the most colored pixels) on a sequence of images. In one embodiment, the optimal image is an image captured at a time corresponding with maximum heart relaxation. Thus, an optimal image may be detected based on capture time of the images compared with, for example, measurements of electrical activity of the heartbeat (e.g., ECG printout) of the patient. In other embodiments, an optimal image may be selected by inputting angiogram video frames featuring a vessel full of contrast agent to a ML model trained to find a significant point of interest in the cardiac cycle (e.g., for example, an R-peak) to select a frame captured at the diastole phase. The trained ML model assists in selecting a frame captured at the diastole phase.

In one embodiment, processor 702 receives a plurality of consecutive images of a patient's vessels and determines presence and possibly location of a diffuse disease and/or multiple lesion (such as a stenosis) in the vessels in one image from the plurality of images (e.g., by applying a machine learning model on one of the images and applying a classifier on the images, as described above) prior to calculating a total pressure drop value of the vessel and/or determining the contribution of one or more portions of the vessel to the total pressure drop and/or any of the other following steps described herein.

An ML unit (such as ML unit 708) according to an embodiment of the invention, is schematically illustrated in FIG. 7B. In this embodiment, ML unit 708 includes a first machine learning (ML) model 721 to provide a prediction 722 of an intermediate value relating to one or more portion of the blood vessel, based on features 720 related to images of the blood vessel. The features 720 may include, for example, spatial and/or temporal features.

ML unit 708 further includes a second ML model 723 to obtain a prediction 726 of a total pressure drop value of the blood vessel, based on the predicted intermediate value. A processor 724 which is in communication with the second ML model 723 then analyzes calculations of the second ML model 723 to determine a contribution 728 of each portion to the total pressure drop value.

Processor 724 or another processor may calculate a new simulated total pressure drop value 729 of the blood vessel by neutralizing the contribution 728 of the one or more portion to the total pressure drop. Processor 724 may also be in communication with a user interface device (such as devices described herein), which may include a monitor or other display, to display to a user a possible treatment strategy which may include information regarding the one or more portion, such as, an indication of the one or more portion and possibly, the new simulated total pressure drop value.

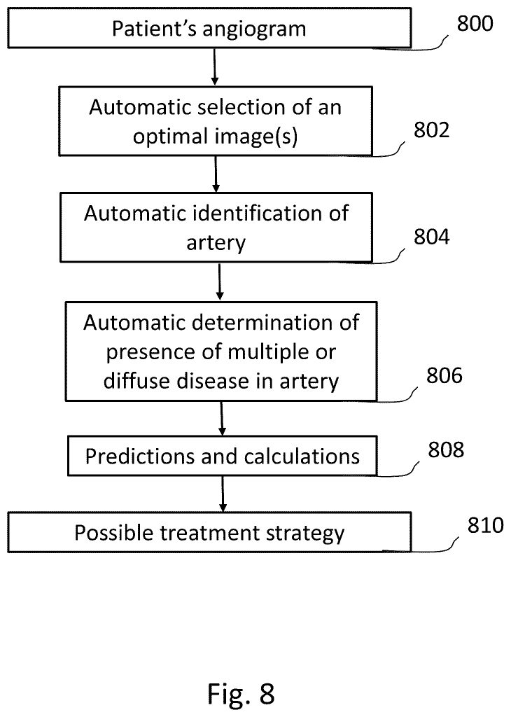

The methods and systems described herein may provide, in concert, a fully automated solution, starting from a patient's angiogram (800) and ending with a possible treatment strategy (810) for multiple or diffuse disease. As schematically illustrated in FIG. 8, a fully automated process, which does not require user input at any stage, may include automatic selection of an optimal image(s) (802) from a patient's angiogram (800), e.g., selection of an image captured at a time corresponding with maximum heart relaxation, as described with reference to FIG. 6 above. Arteries in the optimal image are automatically identified (804) and fully mapped (e.g., as described with reference to FIG. 5 above) so that they may be tracked and so that the presence of multiple or diffuse disease in the artery may be automatically determined (806) from the optimal image(s), as described with reference to FIG. 4 above. A determination of the presence of multiple or diffuse disease may trigger the performance of predictions and calculations (808) such as described above with reference to FIG. 7A and 7B, and a possible treatment strategy may be provided to a user (810), based on the triggered calculations.

In some embodiments, the steps described above may use user input, e.g., for selection of images and/or for identifying arteries and/or pathologies.

The methods described herein may be performed on-line, during angiography, stenting or another procedure on a patient, as well as off-line, in a physician's or technician's office, using data previously collected during the procedure, e.g., using a DICOM file, as described herein.

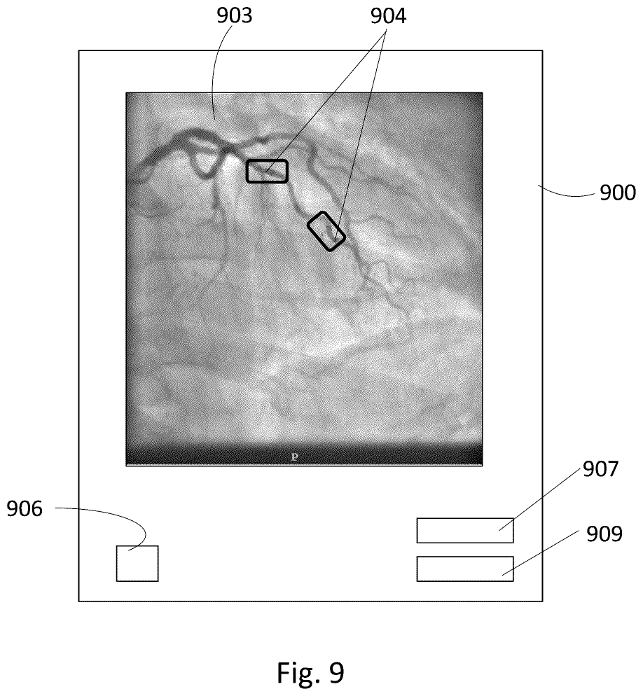

A display, according to one embodiment of the invention, is schematically illustrated in FIG. 9.

Display 900 is part of a user interface device which can be in communication with a processor, such as the processors described herein. Display 900 is configured to exhibit an image 903 of an artery having one or both of diffuse disease and multiple lesions, e.g., an angiogram image obtained via the processor. The image may be an image obtained on-line, e.g., during a procedure and/or an image displayed off-line, e.g., during analysis in the physician's office after the procedure. The image may be accompanied by additional information such as information about the patient.

Also exhibited on the display is an indication 904 of one or more a portion(s) of the artery whose contribution to a total pressure drop value of the artery, when neutralized, changes the total pressure drop value to a value that is within a predetermined range (e.g., within a physiologically healthy range). The indication 904 may provide information to a user regarding which portions of the displayed artery should be treated in order to change the total pressure drop in the artery such that it is within a physiologically healthy range. Namely, which portions of an artery should be treated in order to obtain an artery showing a normal, healthy, total pressure drop. Thus, display 900 may provide a possible treatment strategy for the artery.

In some embodiments, the display includes a button 906 to enable user input of a desired or requested range. The button 906 may be a virtual button and/or may include graphical elements. Thus, a user can input a new total pressure drop value and/or options of portions whose contribution to a total pressure drop value of the artery should be checked, e.g., to calculate if a contribution of a portion input by the user, when neutralized, changes the total pressure drop value to a value that is e.g., equal to or above the inputted value.

In some embodiments, the total pressure drop value 907 of the artery is displayed and/or a new simulated total pressure drop value 909 may be displayed on display 900.

In some embodiments, the image 903 may include a graphical representation of the artery, rather than an actual angiogram image.

In some embodiments, the image 903 includes a cluster of images featuring a same pathology, each of the images in the cluster being captured from a different angle. The cluster of images may be combined to a single image data representation. In other embodiments the image 903 or cluster of images may include a sequence or group of images, each being captured from a different angle.

The indication 904, which may include, for example, graphics, such as, letters, numerals, symbols, different colors and shapes, etc., may be superimposed on the image 903.

Use of display 900 and methods and systems such as described herein, automatically provides information and enables a user to simply and easily decide a treatment strategy (e.g., stenting) for a blood vessel with multiple or diffuse lesions. Since the calculations and resulting information can be done and displayed to a user almost immediately upon receiving images (e.g., angiograms), the decision regarding a treatment strategy (e.g., which vessel to stent) may be taken on-line, during a procedure (e.g., angiography). Additionally, a system, according to embodiments of the invention, may receive as input files that include the images (e.g., angiograms), such as DICOM files, and may perform analysis on the images from the files, enabling to determine a treatment strategy off-line, as well.

What is claimed is:

1. A method for non-invasively determining a treatment strategy for a blood vessel, the method comprising:
obtaining a prediction of a total pressure drop value in the blood vessel based on features generated from a plurality of images of the blood vessel, the images representing all anatomic parts of the blood vessel;
calculating a contribution of one or more portions of the blood vessel to the total pressure drop value;
based on the calculated contribution, calculating a new simulated total pressure drop value of the blood vessel by neutralizing the contribution of the one or more portions to the total pressure drop value; and
displaying to the user information regarding the one or more portions.

2. The method of claim 1, wherein the information comprises an indication of the one or more portions and the new simulated total pressure drop value, the method further comprising displaying to the user the information if the new simulated total pressure drop is determined to be within a physiologically healthy range.

3. The method of claim 1, further comprising:
determining which portion of the one or more portions has a most significant contribution to the total pressure drop; and
neutralizing the contribution of the determined portion to the total pressure drop value.

4. The method of claim 1, wherein each of the plurality of images is captured from a different angle.

5. The method of claim 1, further comprising identifying the blood vessel in each of the plurality of images to track same blood vessels through the plurality of images.

6. The method of claim 5, further comprising identifying the blood vessel in each of the plurality of images and mapping a full path of a predetermined portion of the vessel in each image.

7. The method of claim 6, wherein the predetermined portion of the vessel comprises a portion from a beginning of a left main coronary artery (LMCA) until after bifurcations of the artery.

8. The method of claim 5, further comprising:
using data from the images to create a single data representation; and
extracting features from the single data representation, the features for obtaining the prediction of the total pressure drop value.

9. The method of claim 1, wherein the features comprise spatial and/or temporal features.

10. The method of claim 1, further comprising:
obtaining, from a first machine learning (ML) model, a prediction of an intermediate value relating to the one or more portions of the blood vessel;
inputting the intermediate value to a second ML model to obtain the prediction of the total pressure drop value of the blood vessel; and
analyzing calculations of the second ML model to determine the contribution of each portion to the total pressure drop value.

11. The method of claim 1, wherein the one or more portions of the blood vessel comprises a healthy part of the blood vessel and a part comprising at least one pathology.

12. The method of claim 1, further comprising:
tracking the blood vessel through the plurality of images, each of the images capturing the vessel from a different angle; and
determining presence of diffuse disease or multiple lesions in the blood vessel prior to obtaining the prediction of the total pressure drop value of the blood vessel.

13. The method of claim 1, wherein the plurality of images comprises angiogram images, the method further comprising:
determining which frames in an angiogram video of a patient were captured at a diastole phase of a cardiac cycle of the patient; and
using the frames captured at a diastole phase of a cardiac cycle of the patient, from a plurality of angiogram videos, each captured from a different angle, as the plurality of images.

14. The method of claim 13, wherein determining which frames were captured at the diastole phase, the method further comprising:
selecting frames from the angiogram video of the patient, the frames featuring a vessel full of contrast agent; and
inputting the frames to a ML model trained to find a significant point of interest in the cardiac cycle based on frames featuring a vessel full of contrast agent, each of the frames captured from a different angle, to select a frame captured at the diastole phase.

15. The method of claim 14, wherein the significant point of interest in the cardiac cycle comprises a R-peak as determined by ECG.

16. A system for non-invasively determining a treatment strategy for a blood vessel with multiple or diffuse lesions, the system comprising:
a first machine learning (ML) model to provide a prediction of an intermediate value relating to one or more portion of the blood vessel, based on features related to images of the blood vessel;
a second ML model to obtain a prediction of a total pressure drop value of the blood vessel, based on the intermediate measurement; and
a processor to analyze calculations of the second ML model to determine a contribution of each portion to the total pressure drop value.

17. The system of claim 16, wherein the processor calculates a new simulated total pressure drop value of the blood vessel by neutralizing the contribution of the one or more portion to the total pressure drop value.

18. The system of claim 16, wherein the features comprise spatial and/or temporal features.

19. The system of claim 16, further comprising a display to display to a user information about the one or more portion.

20. A display of a user interface device, the user interface device in communication with a processor, the display configured to exhibit:
an image of an artery having one or both of diffuse disease and multiple lesion; and
an indication of a portion of the artery whose contribution to a total pressure drop value of the artery, when neutralized, changes the total pressure drop value to a value that is within a physiologically healthy range.
